SEQUENCE LISTING (10 sequences)

SEQ ID NO: 1 - protein, 315 aa, synthetic construct
CSTQLGMEGGAIADSQISASSVYMGFMGLQRWGPELARLYRTGIVNAWTASNYDSKPIQVNLLRKMRVSGVMTQGASRAGRAEYLKTFKVAYSLDGRKFEFIQDESGGDKEFLGNLDNNSLKVNMFNPTLEAQYIRLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 2 - protein, 51 aa, synthetic construct
TTNPGVSAWQVNTAYTAGQLVTYNGKTYKCLQPHTSLAGWEPSNVPALWQL

SEQ ID NO: 3 - protein, 197 aa, synthetic construct
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSH

SEQ ID NO: 4 - protein, 563 aa, synthetic construct
CSTQLGMEGGAIADSQISASSVYMGFMGLQRWGPELARLYRTGIVNAWTASNYDSKPIQVNLLRKMRVSGVMTQGASRAGRAEYLKTFKVAYSLDGRKFEFIQDESGGDKEFLGNLDNNSLKVNMFNPTLEAQYIRLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGCCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHTTNPGVSAWQVNTAYTAGQLVTYNGKTYKCLQPHTSLAGWEPSNVPALWQL

SEQ ID NO: 5 - DNA, 945 bp, synthetic construct
  source 1..945: mol_type = other DNA
tgttctacacagctgggcatggaagggggcgccattgctgattcacagatttccgcctcgtctgtgtatatgggtttcatgggcttgcagcgctggggcccggagctggctcgtctgtaccgcacagggatcgtcaatgcctggacagccagcaactatgatagcaagccctggatccaggtgaaccttctgcggaagatgcgggtatcaggtgtgatgacgcagggtgccagccgtgccgggagggcggagtacctgaagaccttcaaggtggcttacagcctcgacggacgcaagtttgagttcatccaggatgaaagcggtggaaacagaggttttttgggtaacctggacaacaacagcctgaaggttaacatgttcaacccgactctggaggcacagtacataaggctgtaccctgttcgtgccaccgcggctgcaccctccgcttcgagctcctgggctgtgagttgcacggatgttctgagcccctgggcctgaagaataacacaattcctgacagccagatgtcagcctccagcagctacaagacatggaacctgcgtgcttttggctggtacccccacttgggaaggctggataatcagggcaagatggatcccctggacggctcagagcaacagtgcccgatggctcaggttgacctgggcactcagaggcaagtgacaggatatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgt

SEQ ID NO: 6 - DNA, 152 bp, synthetic construct
  source 1..152: mol_type = other DNA
acaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttca

SEQ ID NO: 7 - DNA, 556 bp, synthetic construct
  source 1..556: mol_type = other DNA
tgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgtgtttggtcgacgtcgccggggtgccgaccctgctgtggaagctgatcgacgaaatcaagccgggcgattacgcggtgattcaacgcagcgcattcagcgtcgactgtgcagctgcccgcgggaaacccgaatttgcgcccacaacctacacagtcggcgtccctggactggtgcgtttctggaagcacaccaccgagacccggacgcccaagctatcgccgacgagctgaccgacgggcggttctactacgcgaaagtcgccagtgtcaccgacgcgggcgtgcagccggtgtatagccttcgtgtcgacacggcagaccacgc

SEQ ID NO: 8 - DNA, 1653 bp, synthetic construct
  source 1..1653: mol_type = other DNA
tgttctacacagctgggcatggaagggggcgccattgctgattcacagatttccgcctcgtctgtgtatatgggtttcatgggcttgcagcgctggggcccggagctggctcgtctgtaccgcacagggatcgtcaatgcctggacagccagcaactatgatagcaagccctggatccaggtgaaccttctgcggaagatgcgggtatcaggtgtgatgacgcagggtgccagccgtgccgggagggcggagtacctgaagaccttcaaggtggcttacagcctcgacggacgcaagtttgagttcatccaggatgaaagcggtggaaacagaggttttttgggtaacctggacaacaacagcctgaaggttaacatgttcaacccgactctggaggcacagtacataaggctgtaccctgttcgtgccaccgcggctgcaccctccgcttcgagctcctgggctgtgagttgcacggatgttctgagcccctgggcctgaagaataacacaattcctgacagccagatgtcagcctccagcagctacaagacatggaacctgcgtgcttttggctggtacccccacttgggaaggctggataatcagggcaagatggatcccctggacggctcagagcaacagtgcccgatggctcaggttgacctgggcactcagaggcaagtgacaggatatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgttgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgtgtttggtcgacgtcgccggggtgccgaccctgctgtggaagctgatcgacgaaatcaagccgggcgattacgcggtgattcaacgcagcgcattcagcgtcgactgtgcagctgcccgcgggaaacccgaatttgcgcccacaacctacacagtcggcgtccctggactggtgcgtttctggaagcacaccaccgagacccggacgcccaagctatcgccgacgagctgaccgacgggcggttctactacgcgaaagtcgccagtgtcaccgacgcgggcgtgcagccggtgtatagccttcgtgtcgacacggcagaccacgcacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttca

SEQ ID NO: 9 - DNA, 38 bp, synthetic construct
  source 1..38: mol_type = other DNA
attaatcatatgtgttctacacagctgggcatggaagg

SEQ ID NO: 10 - DNA, 35 bp, synthetic construct
  source 1..35: mol_type = other DNA
attaatgctcttccgcaacagcccagcagctccag

(19) United States
(12) Patent Application Publication
(10) Pub. No.: US 2026/0109943 A1
(43) Pub. Date: Apr. 23, 2026

(54) PARTICLE-BASED SEPARATION OF EXTRACELLULAR VESICLES

(71) Applicant: University of Idaho, Moscow, ID (US)

(72) Inventor: Ching-An Peng, Moscow, ID (US)

(73) Assignee: University of Idaho, Moscow, ID (US)

(21) Appl. No.: 19/362,156

(22) Filed: Oct. 17, 2025

Related U.S. Application Data
(60) Provisional application No. 63/708,321, filed on Oct. 17, 2024.

(51) Int. Cl. C12N 5/04; C07K 14/46

(52) U.S. Cl. CPC C12N 5/04; C07K 14/46; C07K 2319/00

(57) ABSTRACT

Provided herein are methods, compositions, and kits for isolating extracellular vesicles (EVs). These methods and systems achieve EV isolation more efficiently, at lower cost, and with higher purity and quality of the isolated EVs, as compared to prior technologies. The disclosed methods include contacting a sample with a chitin-based stationary phase material functionalized with a fusion protein, wherein the fusion protein comprises i) a lactadherin C1C2 domain, ii) a chitin-binding domain (CBD), and iii) an intein positioned between the lactadherin C1C2 domain and the CBD.

### CROSS REFERENCE TO RELATED APPLICATION

This application claims the benefit of U.S. Provisional Application No. 63/708,321, filed Oct. 17, 2024, which is hereby incorporated by reference in its entirety.

### STATEMENT OF GOVERNMENT SUPPORT

This invention was made with government support under Award No. 2022-67022-36600 awarded by USDA-NIFA. The government has certain rights in the invention.

### FIELD

This application relates to the field of extracellular vesicle isolation.

### INCORPORATION OF SEQUENCE LISTING

The Sequence Listing is submitted as an XML file in the form of the file named “7832-112786-02_SequenceListing.xml” (14,192 bytes), which was created on Oct. 16, 2025 which is incorporated by reference herein.

### BACKGROUND

Extracellular vesicles (EVs) play a critical role in a wide range of biological processes, including intercellular communication, immune modulation, and tissue repair. There are several methods for isolating EVs from biological samples, including ultracentrifugation (UC), density gradient centrifugation (DGC), and immunocapture (IC). Each method has its advantages and limitations. The ultracentrifugation method involves spinning the sample at high speeds to pellet EVs and is widely used, but it can co-pellet non-EV particles, resulting in low recovery rates, and is time-consuming. The density gradient centrifugation method involves layering the sample onto a density gradient and centrifuging to separate EVs based on their densities. Although it can separate different types of EVs, it is more time-consuming and requires more specialized equipment. The hydrophilic polymer precipitation method is used for the isolation of EVs from biological fluids by the addition of a hydrophilic polymer, such as polyethylene glycol (PEG), but it may result in co-precipitation of non-EV proteins, lipoproteins, and other macromolecules, leading to contamination of the EVs, and the retention of the polymer may interfere with downstream applications such as proteomics and RNA sequencing. The immunocapture method uses antibodies or aptamers to capture specific EV subtypes from the sample, but it may miss other EV subtypes, such as cancer EVs that don't express CD63 on their surfaces.

There is a need for improved EV isolation methods that address the above limitations.

### SUMMARY

Provided herein are methods, compositions, and kits for isolating extracellular vesicles (EVs). These methods and systems achieve EV isolation more efficiently, less costly, and with higher purity and quality of the isolated EVs, as compared to prior technologies.

Provided herein are methods of isolating extracellular vesicles (EVs) from a sample, including contacting the sample with a chitin-based stationary phase material functionalized with a fusion protein, wherein the fusion protein includes i) a lactadherin C1C2 domain, ii) a chitin-binding domain (CBD), and iii) an intein positioned between the lactadherin C1C2 domain and the CBD. Also provided are fusion proteins including i) a lactadherin C1C2 domain, ii) a chitin-binding domain (CBD), and iii) an intein positioned between the lactadherin C1C2 domain and the CBD. Also provided are nucleic acids and vectors encoding the fusion proteins, as well as chitin-based material functionalized with the fusion proteins. Further provided are kits including the fusion protein, and a chitin-based material; or a chitin-based material functionalized with the fusion protein.

The foregoing and other objects, features, and advantages of the invention will become more apparent from the following detailed description, which proceeds with reference to the accompanying figures.

### SEQUENCE

The nucleic and amino acid sequences listed herein are shown using standard letter abbreviations for nucleotide bases and amino acids. Only one strand of each nucleic acid sequence is shown, but the complementary strand is understood as included by any reference to the displayed strand.

SEQ ID NO: 1 is an exemplary amino acid sequence of a lactadherin C1C2 domain (lactC1C2):

CSTQLGMEGGAIADSQISASSVYMGFMGLQRWGPELARLYRTGIV
NAWTASNYDSKPIQVNLLRKMRVSGVMTQGASRAGRAEYLKTFKV
AYSLDGRKFEFIQDESGGDKEFLGNLDNNSLKVNMFNPTLEAQYI
RLYPVSCHRGCTLRFELLGCELHGCSEPLGLKNNTIPDSQMSASS
SYKTWNLRAFGWYPHLGRLDNQGKINAWTAQSNSAKEWLQVDLGT
QRQVTGIITQGARDFGHIQYVASYKVAHSDDGVQWTVYEEQGSSK
VFQGNLDNNSHKKNIFEKPFMARYVRVLPVSWHNRITLRLELLGC

SEQ ID NO: 2 is an exemplary amino acid sequence of a chitin-binding domain (CBD):

TTNPGVSAWQVNTAYTAGQLVTYNGKTYKCLQPHTSLAGWEPSNV
PALWQL

SEQ ID NO: 3 is an exemplary amino acid sequence of an intein:

CITGDALVALPEGESVRIADIVPGARPNSDNAIDLKVLDRHGNPV
LADRLFHSGEHPVYTVRTVEGLRVTGTANHPLLCLVDVAGVPTLL
WKLIDEIKPGDYAVIQRSAFSVDCAGFARGKPEFAPTTYTVGVPG
LVRFLEAHHRDPDAQAIADELTDGRFYYAKVASVTDAGVQPVYSL
RVDTADHAFITNGFVSH

SEQ ID NO: 4 is an exemplary amino acid sequence of a lactC1C2-intein-CBD fusion protein:

CSTQLGMEGGAIADSQISASSVYMGFMGLQRWGPELARLYRTGIV
NAWTASNYDSKPIQVNLLRKMRVSGVMTQGASRAGRAEYLKTFKV
AYSLDGRKFEFIQDESGGDKEFLGNLDNNSLKVNMFNPTLEAQYI
RLYPVSCHRGCTLRFELLGCELHGCSEPLGLKNNTIPDSQMSASS
SYKTWNLRAFGWYPHLGRLDNQGKINAWTAQSNSAKEWLQVDLGT
QRQVTGIITQGARDFGHIQYVASYKVAHSDDGVQWTVYEEQGSSK
VFQGNLDNNSHKKNIFEKPFMARYVRVLPVSWHNRITLRLELLGC
CITGDALVALPEGESVRIADIVPGARPNSDNAIDLKVLDRHGNPV
LADRLFHSGEHPVYTVRTVEGLRVTGTANHPLLCLVDVAGVPTLL
WKLIDEIKPGDYAVIQRSAFSVDCAGFARGKPEFAPTTYTVGVPG
LVRFLEAHHRDPDAQAIADELTDGRFYYAKVASVTDAGVQPVYSL
RVDTADHAFITNGFVSHTTNPGVSAWQVNTAYTAGQLVTYNGKTY
KCLQPHTSLAGWEPSNVPALWQL

SEQ ID NO: 5 is an exemplary nucleic acid sequence encoding a lactC1C2:

TGTTCTACACAGCTGGGCATGGAAGGGGGCGCCATTGCTGATTCA
CAGATTTCCGCCTCGTCTGTGTATATGGGTTTCATGGGCTTGCAG
CGCTGGGGCCCGGAGCTGGCTCGTCTGTACCGCACAGGGATCGTC
AATGCCTGGACAGCCAGCAACTATGATAGCAAGCCCTGGATCCAG
GTGAACCTTCTGCGGAAGATGCGGGTATCAGGTGTGATGACGCAG
GGTGCCAGCCGTGCCGGGAGGGCGGAGTACCTGAAGACCTTCAAG
GTGGCTTACAGCCTCGACGGACGCAAGTTTGAGTTCATCCAGGAT
GAAAGCGGTGGAAACAGAGGTTTTTTGGGTAACCTGGACAACAAC
AGCCTGAAGGTTAACATGTTCAACCCGACTCTGGAGGCACAGTAC
ATAAGGCTGTACCCTGTTCGTGCCACCGCGGCTGCACCCTCCGCT
TCGAGCTCCTGGGCTGTGAGTTGCACGGATGTTCTGAGCCCCTGG
GCCTGAAGAATAACACAATTCCTGACAGCCAGATGTCAGCCTCCA
GCAGCTACAAGACATGGAACCTGCGTGCTTTTGGCTGGTACCCCC
ACTTGGGAAGGCTGGATAATCAGGGCAAGATGGATCCCCTGGACG
GCTCAGAGCAACAGTGCCCGATGGCTCAGGTTGACCTGGGCACTC
AGAGGCAAGTGACAGGATATCATCACCCAGGGGGCCCGTGACTTT
GGCCACATCCAGTATGTGGCGTCCTACAAGGTAGCCCACAGTGAT
GATGGTGTGCAGTGGACTGTATATGAGGAGCAAGGAAGCAGCAAG
GTCTTCCAGGGCAACTTGGACAACAACTCCCACAAGAAGAACATC
TTCGAGAAACCCTTCATGGCTCGCTACGTGCGTGTCCTTCCAGTG
TCCTGGCATAACCGCATCACCCTGCGCCTGGAGCTGCTGGGCTGT

SEQ ID NO: 6 is an exemplary nucleic acid sequence encoding a CBD:

ACAAATCCTGGTGTATCCGCTTGGCAGGTCAACACAGCTTATACT
GCGGGACAATTGGTCACATATAACGGCAAGACGTATAAATGTTTG
CAGCCCCACACCTCCTTGGCAGGATGGGAACCATCCAACGTTCCT
GCCTTGTGGCAGCTTCA

SEQ ID NO: 7 is an exemplary nucleic acid sequence encoding an intein:

TGCATCACGGGAGATGCACTAGTTGCCCTACCCGAGGGCGAGTCG
GTACGCATCGCCGACATCGTGCCGGGTGCGCGGCCCAACAGTGAC
AACGCCATCGACCTGAAAGTCCTTGACCGGCATGGCAATCCCGTG
CTCGCCGACCGGCTGTTCCACTCCGGCGAGCATCCGGTGTACACG
GTGCGTACGGTCGAAGGTCTGCGTGTGACGGGCACCGCGAACCAC
CCGTTGTGTTTGGTCGACGTCGCCGGGGTGCCGACCCTGCTGTGG
AAGCTGATCGACGAAATCAAGCCGGGCGATTACGCGGTGATTCAA
CGCAGCGCATTCAGCGTCGACTGTGCAGCTGCCCGCGGGAAACCC
GAATTTGCGCCCACAACCTACACAGTCGGCGTCCCTGGACTGGTG
CGTTTCTGGAAGCACACCACCGAGACCCGGACGCCCAAGCTATCG
CCGACGAGCTGACCGACGGGCGGTTCTACTACGCGAAAGTCGCCA
GTGTCACCGACGCGGGCGTGCAGCCGGTGTATAGCCTTCGTGTCG
ACACGGCAGACCACGC

SEQ ID NO: 8 is an exemplary nucleic acid sequence encoding a lactC1C2-intein-CBD fusion protein:

TGTTCTACACAGCTGGGCATGGAAGGGGGCGCCATTGCTGATTCA
CAGATTTCCGCCTCGTCTGTGTATATGGGTTTCATGGGCTTGCAG
CGCTGGGGCCCGGAGCTGGCTCGTCTGTACCGCACAGGGATCGTC
AATGCCTGGACAGCCAGCAACTATGATAGCAAGCCCTGGATCCAG
GTGAACCTTCTGCGGAAGATGCGGGTATCAGGTGTGATGACGCAG
GGTGCCAGCCGTGCCGGGAGGGCGGAGTACCTGAAGACCTTCAAG
GTGGCTTACAGCCTCGACGGACGCAAGTTTGAGTTCATCCAGGAT
GAAAGCGGTGGAAACAGAGGTTTTTTGGGTAACCTGGACAACAAC
AGCCTGAAGGTTAACATGTTCAACCCGACTCTGGAGGCACAGTAC
ATAAGGCTGTACCCTGTTCGTGCCACCGCGGCTGCACCCTCCGCT
TCGAGCTCCTGGGCTGTGAGTTGCACGGATGTTCTGAGCCCCTGG
GCCTGAAGAATAACACAATTCCTGACAGCCAGATGTCAGCCTCCA
GCAGCTACAAGACATGGAACCTGCGTGCTTTTGGCTGGTACCCCC
ACTTGGGAAGGCTGGATAATCAGGGCAAGATGGATCCCCTGGACG
GCTCAGAGCAACAGTGCCCGATGGCTCAGGTTGACCTGGGCACTC
AGAGGCAAGTGACAGGATATCATCACCCAGGGGGCCCGTGACTTT
GGCCACATCCAGTATGTGGCGTCCTACAAGGTAGCCCACAGTGAT
GATGGTGTGCAGTGGACTGTATATGAGGAGCAAGGAAGCAGCAAG
GTCTTCCAGGGCAACTTGGACAACAACTCCCACAAGAAGAACATC
TTCGAGAAACCCTTCATGGCTCGCTACGTGCGTGTCCTTCCAGTG
TCCTGGCATAACCGCATCACCCTGCGCCTGGAGCTGCTGGGCTGT
TGCATCACGGGAGATGCACTAGTTGCCCTACCCGAGGGCGAGTCG
GTACGCATCGCCGACATCGTGCCGGGTGCGCGGCCCAACAGTGAC
AACGCCATCGACCTGAAAGTCCTTGACCGGCATGGCAATCCCGTG
CTCGCCGACCGGCTGTTCCACTCCGGCGAGCATCCGGTGTACACG
GTGCGTACGGTCGAAGGTCTGCGTGTGACGGGCACCGCGAACCAC
CCGTTGTGTTTGGTCGACGTCGCCGGGGTGCCGACCCTGCTGTGG
AAGCTGATCGACGAAATCAAGCCGGGCGATTACGCGGTGATTCAA
CGCAGCGCATTCAGCGTCGACTGTGCAGCTGCCCGCGGGAAACCC
GAATTTGCGCCCACAACCTACACAGTCGGCGTCCCTGGACTGGTG
CGTTTCTGGAAGCACACCACCGAGACCCGGACGCCCAAGCTATCG
CCGACGAGCTGACCGACGGGCGGTTCTACTACGCGAAAGTCGCCA
GTGTCACCGACGCGGGCGTGCAGCCGGTGTATAGCCTTCGTGTCG
ACACGGCAGACCACGCACAAATCCTGGTGTATCCGCTTGGCAGGT
CAACACAGCTTATACTGCGGGACAATTGGTCACATATAACGGCAA
GACGTATAAATGTTTGCAGCCCCACACCTCCTTGGCAGGATGGGA
ACCATCCAACGTTCCTGCCTTGTGGCAGCTTCA

SEQ ID NO: 9 and SEQ ID NO: 10 are exemplary primer sequences.

### BRIEF DESCRIPTION OF THE DRAWINGS

FIG. 1A: A plasmid map for pTXB1-lactadherin C1C2 (lactC1C2). The gene encoding lactC1C2 was inserted between the NdeI and SapI sites.

FIG. 1B: An image of an exemplary 1% agarose gel that was used for DNA gel electrophoresis of PCR-amplified lactC1C2 gene (˜0.948 kb) (lane 1), and DNA ladder (lane L).

FIG. 2A: A representative purification profile for lactC1C2 protein purification. OD₂₈₀ was measured for each of the collected fractions.

FIG. 2B: An image of a representative SDS-PAGE gel showing protein markers (lane M) and purified lactC1C2 protein (lane 1).

FIG. 2C: An image of a representative membrane used in Western blot analysis with an anti-lactC1C2 antibody. Lane M: protein markers; lane 1: purified lactC1C2 protein.

FIG. 3A: An image of a representative SDS-PAGE gel. Lane M: protein markers; lane P: Lemo21(DE3) pellets; lane CP: crude bacterial lysates; lane FT: flow-through; lanes E1, E2, and E3: elution fractions of the purified lactC1C2 protein.

FIG. 3B: An image of a representative membrane used in Western blot analysis with an anti-lactC1C2 antibody. Lane M: protein markers; lane P: Lemo21(DE3) pellets; lane CP: crude bacterial lysate; lane FT: flow-through; lanes E1, E2, and E3: elution fractions of the purified lactC1C2 protein.

FIG. 4A: Images of representative membranes used in Western blot analysis with anti-CD63, anti-CD81, anti-Alix, and anti-Tsg101 antibodies, for mesenchymal stem cell (MSC)-derived EVs isolated with chitin magnetic beads functionalized with lactC1C2-intein-CBD fusion protein.

FIG. 4B: Representative transmission electron microscopy (TEM) images of MSC-derived EVs isolated with chitin magnetic beads functionalized with lactC1C2-intein-CBD fusion proteins, as compared to MSC-derived EVs isolated using the protamine sulfate method, and a control.

FIGS. 5A-5F: Characterization of plant EVs isolated by two methods: the present method, which utilizes lactC1C2-functionalized chitin magnetic particles, and the differential ultracentrifugation (DUCG) method, which represents the current gold standard for EV isolation. FIGS. 5A-5B: Graphs showing results of dynamic light scattering (DLS) analysis of EVs isolated by the present method (A) and the DUCG method (B). FIGS. 5C-5D: Graphs showing results of nanoparticle tracking analysis (NTA) of EVs isolated by the present method (C) and the DUCG method (D). FIGS. 5E-5F: Representative TEM images of EVs isolated by the present method (E) and the DUCG method (F).

FIG. 6: Images of representative membranes used in Western blot analysis with anti-CD9, anti-CD81, anti-CD63, anti-TSG101, and anti-calnexin antibodies for plant EVs isolated by the present method (denoted as LactCIC2-EVs), and EVs isolated by the DUCG method (denoted as DUCG-EVs).

FIGS. 7A-7B: Images of representative membranes used in immunoblot analysis with anti-CD63 and anti-lactC1C2 antibodies. The sample used in FIG. 7A was from the first phosphate-buffered saline (PBS) wash, and the sample used in FIG. 7B was from the second PBS wash.

### DETAILED DESCRIPTION

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various aspects, the following explanations of terms are provided.

About: Unless the context indicates otherwise, “about” refers to plus or minus 5% of a reference value. For example, “about” 100 refers to 95 to 105.

Chitin-based stationary phase material: A material comprising a chitin, which can bind to a protein through chitin affinity interaction for protein purification. Chitin refers to a polysaccharide comprising N-acetylglucosamine units linked by β-1,4 bonds. The chitin of the material may be isolated from natural sources or synthesized. The chitin includes unmodified (naturally occurring) or modified chitin. Exemplary modifications include one or more of deacetylation, carbamoylation, etherification, acylation, etc. In some examples, the material may include a solid support matrix, to which the chitin is attached (e.g., through covalent bonding); such a material is also termed chitin resin. The chitin resin is not limited to any particular physical form and may include, for instance, particles, beads, gels, or other porous structures. In some other examples, the material is formed by chitin only (or chitin with reinforcing agents, like crosslinking agents) and does not include any other matrix or supporting material. Such a material can be formed by chitin regeneration, and may include particles, beads, gels, or other porous structures. Chitin beads encompass all chitin-based materials that include discrete particulate or generally spherical structures. In some examples, the material is magnetic, e.g., by comprising a magnetic component.

Chitin-binding domain (CBD): A protein domain that specifically binds to chitin (modified or unmodified), e.g., as in a chitin-based stationary phase material. In some examples, the CBD includes an amino acid sequence that is at least 70%, at least 80%, at least 90%, at least 95%, at least 96%, at least 97%, at least 98%, at least 99%, or at least 99.5% identical to SEQ ID NO: 2, or includes or consists of SEQ ID NO: 2. In some examples, the CBD is encoded by a nucleic acid that includes a nucleic acid sequence that is at least 70%, at least 80%, at least 90%, at least 95%, at least 96%, at least 97%, at least 98%, at least 99%, or at least 99.5% identical to SEQ ID NO: 6, or includes or consists of SEQ ID NO: 6.

Conservative substitution: A substitution of one amino acid residue in a protein sequence for a different amino acid residue having similar biochemical properties. Typically, conservative substitutions have little to no impact on the activity of a resulting polypeptide. For example, variants of a protein domain or fusion protein disclosed herein can include one or more conservative substitutions (for example no less than 1, 2, 3, 4, 5, 6, 7, 8, 9, or 10, and/or no more than 2, 3, 4, 5, 6, 7, 8, 9, 10, 15, 20, 25, 30, 35, 40, 45, or 50 substitutions, for example 2 to 50, 2 to 30, 2 to 20, 2 to 15, 2 to 10, 2 to 5 substitutions) and retain the structure and activity of the original protein. A protein can be produced to contain one or more conservative substitutions by manipulating the nucleotide sequence that encodes the protein using, for example, standard procedures such as site-directed mutagenesis or PCR.

Examples of conservative substitutions are shown below.

| Original | Conservative |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted by a hydrophobic residue, for example, leucyl, isoleucyl, phenylalanyl, valyl or alanyl; (b) a cysteine or proline is substituted by any other residue; (c) a residue having an electropositive side chain, for example, lysyl, arginyl, or histadyl, is substituted by an electronegative residue, for example, glutamyl or aspartyl; or (d) a residue having a bulky side chain, for example, phenylalanine, is substituted by one not having a side chain, for example, glycine.

Extracellular vesicle (EV): Lipid bilayer-delimited particles that are released from a cell, but cannot replicate. EVs range in diameter from about 20 nm to about 10 μm but are typically smaller than 200 nm. EVs can carry proteins, nucleic acids, lipids, metabolites, and/or even organelles from the parent cell. Various types of EVs are included. For example, EVs vary in size, biogenesis pathway, cargo, cellular source, and function. Examples of EVs include exosomes, microvesicles (also known as ectosomes, shed vesicles, or microparticles), and apoptotic bodies.

Intein: A protein segment that can undergo thiol-induced self-cleavage in the presence of a thiol-containing reagent. The term includes both naturally occurring and engineered inteins. In some examples, the intein includes a cysteine or serine at its N-terminus or C-terminus, which can initiate an N—S or N—O acyl shift that converts the amide bond between the intein and the lactC1C2 into a thioester linkage, which can be cleaved by a thiol-containing reagent. In some examples, the intein includes an amino acid sequence that is at least 70%, at least 80%, at least 90%, at least 95%, at least 96%, at least 97%, at least 98%, at least 99%, or at least 99.5% identical to SEQ ID NO: 3, or includes or consists of SEQ ID NO: 3. In some examples, the intein is encoded by a nucleic acid that includes a nucleic acid sequence that is at least 70%, at least 80%, at least 90%, at least 95%, at least 96%, at least 97%, at least 98%, at least 99%, or at least 99.5% sequence identity to SEQ ID NO: 7, or includes or consists of SEQ ID NO: 7.

Isolated or purified: An isolated or purified biological substance (such as a nucleic acid, protein, EV, or cell) has been substantially separated, or produced apart from other biological substances, with which it is naturally associated. An isolated or purified biological substance can be obtained, for example, by isolation or purification from a biological sample, by recombinant expression or production in host cells followed by purification, or by chemical synthesis followed by purification. The term “isolated” or “purified” does not require absolute purity; rather, it is intended as a relative term, for example, referring to being more enriched with (or having a higher concentration of) the substance, compared to a crude preparation or a natural environment from which the substance is isolated or purified. In some aspects, a biological substance is purified if the substance represents at least 50%, at least 60%, at least 70%, at least 80%, at least 90%, at least 95%, at least 96%, at least 97%, at least 98%, at least 99%, at least 99.5%, or greater, of the total moles, mass, or number of particles of non-solvent substances in a preparation.

Lactadherin: Also known as milk fat globule-EGF factor 8 (MFG-E8), a glycoprotein that belongs to the secreted extracellular matrix protein family and is present in many mammalian species and in humans is encoded by the MFGE8 gene. Lactadherin typically includes one or more epidermal growth factor (EGF)-like domains, and C-terminal C1 and C2 domains (collectively referred to as the C1C2 domain) that are homologous to those of coagulation factors V and VIII. The C1C2 domain can specifically bind to phosphatidylserine (PS). In some examples, the lactadherin C1C2 domain (lactC1C2) includes an amino acid sequence that is at least 70%, at least 80%, at least 90%, at least 95%, at least 96%, at least 97%, at least 98%, at least 99%, or at least 99.5% identical to SEQ ID NO: 1, or includes or consists of SEQ ID NO: 1. In some examples, the lactC1C2 is encoded by a nucleic acid that includes a nucleic acid sequence that is at least 70%, at least 80%, at least 90%, at least 95%, at least 96%, at least 97%, at least 98%, at least 99%, or at least 99.5% identical to SEQ ID NO: 5, or includes or consists of SEQ ID NO: 5.

Nucleic acid: A polymeric form of nucleotides, synonymous with polynucleotide. A nucleotide refers to a monomer that includes a base linked to a sugar (such as a pyrimidine, purine, or synthetic analogs thereof), or a base linked to an amino acid (e.g., as in a peptide nucleic acid). Nucleotides include ribonucleotides, deoxyribonucleotides, related naturally occurring structural variants, and synthetic non-naturally occurring analogs thereof. A nucleotide is one monomer in a polynucleotide. A nucleotide sequence refers to the sequence of bases in a polynucleotide. Nucleic acid molecules include DNA, RNA, and complexes of DNA and RNA. A nucleic acid molecule may be linear, circular, single-stranded, double-stranded, or partially double stranded. A double-stranded or partially double-stranded nucleic acid molecule is typically formed by two strands of polynucleotides held together through hydrogen bonding between complementary nucleobases. Nucleic acid can refer to a complete molecule, or any segment or portion of the complete molecule. It will be understood that when a nucleotide sequence is represented by a DNA sequence (e.g., A, T, G, C), this also includes an RNA sequence (e.g., A, U, G, C) in which “U” replaces “T,” and vice versa. A recombinant nucleic acid molecule refers to a nucleic acid molecule having nucleotide sequences that are not naturally joined together.

Operably linked: A first nucleic acid is operably linked to a second nucleic acid when the first nucleic acid is placed in a functional relationship with the second nucleic acid. In some examples, operably linked DNA sequences are contiguous and, where necessary to join two protein coding regions, in the same reading frame. In some examples, operably linked nucleic acids include a first nucleic acid contiguous with the 5′ or 3′ end of a second nucleic acid. In some examples, one or more regulatory elements (e.g., promoters, enhancers, etc.) are operably linked to a nucleic acid sequence when they control the expression of the sequence.

Protein: A polymer formed by two or more amino acids, regardless of length or post-translational modification (e.g., glycosylation or phosphorylation). Peptide, polypeptide, and protein are used interchangeably herein. A fusion protein refers to a protein that includes parts (e.g., domains) from two or more different proteins, which can be produced from a nucleic acid molecule formed by joining the coding sequences of the parts together.

Recombinant: Of or resulting from new combinations of genetic material or amino acid sequences. A recombinant protein is typically a protein produced by the use of recombinant DNA technology, which involves the combination of genetic material from different sources to create a new (non-naturally occurring) DNA molecule, which is then introduced into a host organism (such as bacteria, yeast, or mammalian cells) to produce the desired protein.

Sample: Any material of analytical interest. A sample may be obtained from an organism (e.g., a human, animal, plant, or microorganism), a natural environment (e.g., soil, water, or air), an artificial or controlled environment (e.g., a cell or tissue culture, bioreactors, or other industrial or laboratory systems used to grow or maintain biological materials). A biological sample is a sample that includes a biological substance, such as biomolecules (e.g., nucleic acids, proteins, lipids, and polysaccharides), EVs, or cells. Common types of biological samples include bodily fluids (e.g., blood, derivatives and fractions of blood (e.g., serum, plasma, etc.), urine, saliva, cerebrospinal fluid, amniotic fluid, prostate fluid, tears, milk, mucus, sputum, etc.), surface washings, nasal washings, swabs and scrapes, fine-needle aspirates, bone marrow aspirates, liquid biopsy samples, solid biopsy samples, culture media, etc. The term biological sample encompasses both primary samples (which are materials directly collected from a source) and processed samples (which are samples that have undergone one or more deliberate steps to enrich a target, enhance a signal from a target, or otherwise assist with the detection of a target, such as by isolation, purification, amplification, modification, etc.).

Sequence identity: The identity between two or more nucleic acid molecules, or two or more proteins, is expressed in terms of the percentage identity between the sequences. Sequence identity can be measured in terms of percentage identity; the higher the percentage, the more identical the sequences. Homologs, orthologs, or variants of a protein or nucleic acid will possess a relatively high degree of sequence identity when aligned using standard methods. Similar functions can be inferred based on homology, which can in turn be inferred from sequence identity.

Any suitable method may be used to align sequences for comparison. Non-limiting examples of programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics, 5(2):151-3, 1989; Corpet, Nucleic Acids Res. 16(22):10881-10890, 1988; Huang et al. Bioinformatics, 8(2):155-165, 1992; and Pearson, Methods Mol. Biol. 24:307-331, 1994., Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the full length of the reference sequence, or by an articulated length (such as 100 consecutive nucleotide or amino acid residues from the reference sequence), followed by multiplying the resulting value by 100.

Homologs and variants of a protein or nucleic acid are typically characterized by possession of at least about 75% sequence identity, such as at least about 80%, 85%, 90%, 91%, 92%, 93%, 94%, 95%, 96%, 97%, 98% or 99% sequence identity, counted over the full-length of the protein or nucleic acid.

Thiol-containing reagent: A compound including one or more thiol (—SH) groups that can act as a nucleophile to induce cleavage of an intein. Exemplary thiol-containing reagents include dithiothreitol (DTT), β-mercaptoethanol, 2-mercaptoethanesulfonate (MESNA), and cysteine.

Vector: A delivery system includes a nucleic acid molecule (e.g., DNA or RNA) carrying a sequence of interest, and optionally includes other components. The nucleic acid molecule can be single-stranded, double-stranded, or partially double-stranded, and can comprise one or more free ends, or no free ends (e.g., circular). A vector can transduce or transfect a host cell and permit expression and/or integration of the nucleic acid sequence of interest in the host cell. A vector may also be capable of replicating in a host cell (e.g., along with or independent of the host genome replication during cell division), for example, by including a nucleic acid sequence (such as an origin of replication) that permits its replication. An integrating vector is capable of integrating the nucleic acid sequence of interest it carries into a host nucleic acid molecule. An expression vector contains necessary regulatory sequences that allow expression (transcription and optionally translation) of the nucleic acid sequence of interest. Plasmid is a type of vectors that is a circular, double-stranded DNA molecule. A viral vector includes viral proteins and optionally a lipid envelope that enclose the nucleic acid molecule, forming a particle. The term viral vector also encompasses the viral nucleic acid carrying the sequence of interest alone, without other viral components, e.g., after the nucleic acid is released into the transduced cell.

### II. Overview

Extracellular vesicles (EVs) are lipid bilayer-enclosed, cytosol-containing vesicles, which are released by cells of eukaryotic and prokaryotic organisms into the extracellular space. EVs can vary in size, content, and function, and include three main types: exosomes, microvesicles, and apoptotic bodies. Exosomes are small vesicles (30-150 nm in diameter) that are derived from the endosomal pathway and are released by exocytosis. Microvesicles (also known as ectosomes, shed vesicles, or microparticles) are larger (100-1000 nm) and are released from the plasma membrane by budding. Apoptotic bodies are larger still, ranging from 1 to 5 micrometers, and are formed during programmed cell death. EVs contain a variety of molecules, including proteins, lipids, nucleic acids (e.g., microRNAs, small RNAs, etc.), and metabolites, and are involved in many biological processes, including intercellular communication, immune modulation, and tissue repair.

EVs have a variety of functions in intercellular communication, immune modulation, and tissue repair. EVs can transfer bioactive molecules, such as proteins, lipids, nucleic acids, and metabolites between cells, allowing for the exchange of information and the coordination of cellular processes. EVs can modulate the immune response by delivering antigens or other signals to immune cells, or by transferring regulatory molecules that suppress or activate immune responses. EVs can promote tissue repair by delivering growth factors, extracellular matrix molecules, and other signaling molecules to damaged tissues. The pathogen defense mechanism of EVs can contribute to the defense against pathogens by delivering antimicrobial peptides, immune molecules, or other molecules that directly or indirectly target pathogens. EVs have been shown to play a role in cancer progression by facilitating the spread of cancer cells to other tissues (metastasis) and by modulating the immune response to the cancer.

EVs possess therapeutic activities that can potentially treat diseases and improve human health. Additionally, EVs represent promising carriers for drug delivery compared to artificially synthesized drug carriers. EVs are also used as disease markers, and considered an important part of a liquid biopsy because circulating EVs reflect systematic health status since all the tissues in the body contribute EVs in circulation.

Plant EVs have been isolated from many plant species and play a prominent role in immune system modulation and plant defense response. Plant-derived EVs (PDEVs) possess therapeutic activities that can potentially treat diseases and improve human health. In addition, PDEVs represent promising carriers for drug delivery compared to artificially synthesized drug carriers, as they are natural products with advantageous properties including safety, non-toxicity, low immunogenicity, and less allergenic nature. Moreover, PDEVs are stable and resistant in the stomach- and intestinal-like solution. Anticancer activities of PDEVs have been investigated and reported to be associated with various mechanisms of action. In general, PDEVs revealed anti-proliferative and pro-apoptotic activities on cancer cells without inducing harmful effects on non-cancerous cells. For example, PDEVs isolated from Citrus limon juice inhibited the proliferation of tumor cell lines (A549, SW480, and LAMA84) without affecting the viability of healthy cells (HS5, HUVEC, and PBMC).

The gold standard procedure for isolation and purification of EVs is differential ultracentrifugation with consecutive steps of low centrifugal forces to remove cellular debris and then high-speed forces to collect EVs based on density. However, the quantity and quality of the EVs obtained can be influenced by several parameters such as centrifugal force, rotor type, and solution viscosity. Moreover, this approach lacks specificity, leading to the co-precipitation of non-EV proteins and other particulate contaminants from disrupted cells. As a result, the products obtained by such separation methods cannot be regarded as rigorous EVs.

Provided herein are novel methods, compositions, and kits for EV isolation, wherein a lactadherin C1C2 (lactC1C2)-containing fusion protein is used to bind EVs through the specific interaction between lactC1C2 and phosphatidylserine (PS) present on EV membrane. The methods and systems of the present disclosure achieve isolation of EVs with significantly enhanced purity and morphology, and with reduced cost.

### II. Fusion Proteins

Provided are fusion proteins including i) a lactadherin C1C2 (lactC1C2) domain, ii) a chitin-binding domain (CBD), and iii) an intein positioned between the lactC1C2 domain and the CBD. These fusion proteins can be used to functionalize a chitin-based material, such as a chitin-based stationary phase material used in chromatography. Functionalization can be achieved by contacting a preparation including the fusion protein with the chitin-based material, followed by removal of the liquid or supernatant, and optionally one or more washes. Crude cell lysates of cells (e.g., E. coli cells) expressing the fusion protein can achieve efficient functionalization, without the need of purification of the fusion protein prior to the functionalization. Thus, any preparation or composition including the fusion protein, purified or not, can achieve efficient functionalization of a chitin-based material.

In some aspects, the lactC1C2 domain of the fusion protein includes an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 1, or includes or consists of SEQ ID NO: 1. In some aspects, the lactC1C2 domain of the fusion protein is encoded by a nucleic acid that includes a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 5, or includes or consists of SEQ ID NO: 5. In some examples, the lactC1C2 domain is no more than 360 amino acids (aa) in length, such as no more than 355, 350, 345, 340, 335, 330, 325, or 320 aa in length, and/or is no less than 270 aa in length, such as no less than 275, 280, 285, 290, 295, 300, 305, or 310 aa in length.

In some aspects, the CBD of the fusion protein includes an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 2, or includes or consists of SEQ ID NO: 2. In some aspects, the CBD of the fusion protein is encoded by a nucleic acid that includes a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 6, or includes or consists of SEQ ID NO: 6. In some examples, the CDB is no more than 70 aa in length, such as no more than 65, 60, or 55 aa in length, and/or is no less than 30 aa in length, such as no less than 35, 40, or 45 aa in length.

In some aspects, the intein of the fusion protein includes an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 3, or includes or consists of SEQ ID NO: 3. In some aspects, the intein of the fusion protein is encoded by a nucleic acid that includes a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 7, or includes or consists of SEQ ID NO: 7. In some examples, the intein is no more than 240 aa in length, such as no more than 235, 230, 225, 220, 215, 210, or 205 aa in length, and/or is no less than 160 aa in length, such as no less than 165, 170, 175, 180, 185, 190, or 195 aa in length.

In some aspects, the fusion protein includes, from N-terminal to C-terminal, the lactC1C2 domain, the intein, and the CBD. In some aspects, the fusion protein includes, from N-terminal to C-terminal, the CBD, the intein, and the lactC1C2 domain. In some aspects, the fusion protein includes only the lactC1C2 domain, the CBD, and the intein. In some aspects, the fusion protein includes an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 4, or includes or consists of SEQ ID NO: 4. In some aspects, the fusion protein is encoded by a nucleic acid that includes a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 8, or includes or consists of SEQ ID NO: 8. In some examples, the fusion protein is no more than 620 aa in length, such as no more than 615, 610, 605, 600, 595, 590, 585, 580, 575, 570 aa in length, and/or is no less than 500 aa in length, such as no less than 505, 510, 515, 520, 525, 530, 535, 540, 545, or 550 aa in length.

### III. Polynucleotides and Expression

Nucleic acid molecules (for example, DNA or RNA molecules) encoding the amino acid sequences of the fusion proteins disclosed herein are provided. Sequences of the nucleic acids encoding these proteins can readily be obtained based on the amino acid sequences provided herein or available in the art, and the genetic code. In several implementations, the nucleic acid molecules can be expressed in a host cell (such as a microbial cell or mammalian cell) to produce a desired protein. The genetic code can be used to construct a variety of functionally equivalent nucleic acid sequences, such as nucleic acids which differ in sequence, but which encode the same fusion protein.

Provided are nucleic acids encoding the fusion proteins disclosed herein. In some examples, the nucleic acid includes a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 8, or includes or consists of SEQ ID NO: 8. Also provided are vectors including the nucleic acids.

The disclosed nucleic acids include DNAs, cDNAs and RNAs. The coding sequence includes variants that result from the degeneracy (e.g., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA, and so on. Tables showing the standard genetic code can be found in various sources (see e.g., Stryer, 1988, Biochemistry, 3rd Edition, W.H. 5 Freeman and Co., NY).

In addition, the disclosed nucleic acids may be codon-optimized for expression in a given organism or a given cell. Codon usage bias, the use of synonymous codons at unequal frequencies, is ubiquitous among genetic systems (Ikemura, J. Mol. Biol. 146:1-21, 1981; Ikemura, J. Mol. Biol. 158:573-97, 1982). The strength and direction of codon usage bias is related to genomic G+C content and the relative abundance of different isoaccepting tRNAs (Akashi, Curr. Opin. Genet. Dev. 11:660-666, 2001; Duret, Curr. Opin. Genet. Dev. 12:640-9, 2002; Osawa et al., Microbiol. Rev. 56:229-264, 1992). Codon usage can affect the efficiency of gene expression. Codon-optimization refers to replacement of at least one codon (such as at least 5 codons, at least 10 codons, at least 25 codons, at least 50 codons, at least 75 codons, at least 100 codons or more) in a nucleic acid sequence with a synonymous codon (one that codes for the same amino acid) more frequently used (preferred) in the organism. Each organism has a particular codon usage bias for each amino acid, which can be determined from publicly available codon usage tables (for example see Nakamura et al., Nucleic Acids Res. 28:292, 2000 and references cited therein). For example, a codon usage database is available at kazusa.or.jp/codon. One of ordinary skill in the art can modify a nucleic acid encoding a particular amino acid sequence, such that it encodes the same amino acid sequence, while being optimized for expression in a particular cell type.

Nucleic acid molecules encoding the fusion protein can be prepared by any suitable method including, for example, cloning of appropriate sequences, or direct chemical synthesis by standard methods. Chemical synthesis produces a single stranded oligonucleotide. This can be converted into double stranded DNA by hybridization with a complementary sequence or by polymerization with a DNA polymerase using the single strand as a template. Nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017). Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect, and mammalian cells. A DNA sequence encoding the fusion protein can be expressed in vitro by DNA transfer into a suitable host cell. The cell may be prokaryotic or eukaryotic. Numerous expression systems available for expression of proteins, including Bacillus anthracis, E. coli, other bacterial hosts, yeast, and various higher eukaryotic cells such as the COS, CHO, HeLa, and myeloma cell lines, can be used to express the disclosed fusion protein. In a particular example, the host cell is an E. coli cell, such as a Lemo21(DE3) E. coli cell. Methods of stable transfer, meaning that the foreign DNA is continuously maintained in the host, are known in the art.

Expression of the fusion proteins disclosed herein can be achieved by operably linking a nucleic acid encoding the fusion protein to a promoter. The promoter can be any promoter of interest, including constitutive and inducible promoters. An expression cassette suitable for replication and/or integration in either prokaryotes or eukaryotes can be utilized. Typical expression cassettes contain specific sequences useful for regulation of the expression of the protein-encoding DNA. For example, the expression cassettes may include appropriate promoters, enhancers, transcription and translation terminators, initiation sequences, a start codon in front of a protein coding sequence, splicing signals for introns, stop codons, etc. The nucleic acid encoding the fusion protein, or the expression cassette can be part of a vector. The vector can further encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, GPt, neo, and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

### IV. Methods, Compositions, and Kits for Isolating EVs

Provided are methods of isolating EVs from a sample, including contacting the sample with a chitin-based material functionalized with a fusion protein disclosed herein, wherein the fusion protein includes i) a lactC1C2 domain, ii) a CBD, and iii) an intein positioned between the lactC1C2 domain and the CBD.

In some examples, the chitin-based material is a chitin-based stationary phase material used in chromatography. In certain examples, the chitin-based material is chitin beads. In some examples, the chitin-based material is magnetic, e.g., magnetic chitin beads. Methods for making magnetic chitin-based material are known in the art; for example, the methods may include mixing a chitin solution with magnetic nanoparticles (such as Fe₃O₄) to create a uniform suspension, followed by bead formation, or coating pre-formed magnetic cores with a layer of chitin. The chitin-based material is functionalized with the fusion protein via non-covalent interactions between chitin and the CBD domain of the fusion protein, such that the material presents lactC1C2 that can bind specifically and non-covalently to PS.

In some examples, the fusion protein includes, from N-terminal to C-terminal, the lactC1C2 domain, the intein, and the CBD domain. In some examples, the fusion protein includes an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 4, or includes or consists of SEQ ID NO: 4; or is encoded by a nucleic acid that includes a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to SEQ ID NO: 8, or includes or consists of SEQ ID NO: 8.

In some examples, the EVs have a diameter of less than 200 nm. In some examples, the EVs include exosomes, microvesicles, and/or apoptotic bodies. In some examples, the EVs include exosomes. In some examples, the EVs range in diameter from about 30 nm to about 200 nm, such as about 50 nm to about 200 nm, about 30 nm to about 150 nm, about 40 nm to about 150 nm, about 50 nm to about 150 nm, about 60 nm to about 150 nm, about 70 nm to about 150 nm, about 80 nm to about 150 nm, about 90 nm to about 150 nm, about 100 nm to about 150 nm, about 110 nm to about 150 nm, about 120 nm to about 150 nm, about 110 nm to about 140 nm, or about 110 nm to about 130 nm. In some examples, the sizes are measured by NTA.

Any suitable sample may be used for EV isolation according to the present methods. In some aspects, the sample is a liquid (including solution, suspension, or any other forms of a mixture that includes a liquid). In some examples, the sample is a biological sample.

In some examples, the biological sample is from a human or animal (e.g., non-human mammals), and the methods can be used to isolate human or animal EVs. In some examples, the biological sample is from a veterinary subject. In certain examples, the biological sample is a liquid biopsy (e.g., a bodily fluid).

In some examples, the biological sample is from the media of cultured cells or tissues, and the methods can be used to isolate EVs that originate from the cultured cells or tissues. In some examples, the cultured cells include cultured human cells, mammalian cells, plant cells, or microbial (e.g., bacterium, archaeon, fungus, yeast, mold, protozoan, and alga) cells. In some examples, the cultured cells include stem cells (e.g., totipotent stem cells, pluripotent stem cells, embryonic stem cells, induced pluripotent stem cells, multipotent stem cells, hematopoietic stem cells, mesenchymal stem cells, neural stem cells, endothelial progenitor cells, epithelial stem cells, cardiac stem cells, liver progenitor cells, spermatogonial stem cells, perinatal stem cells, umbilical cord stem cells, placental stem cells, amniotic stem cells, adipose-derived stem cells, bone marrow stem cells, skin stem cells, muscle satellite cells, cancer stem cells, equine mesenchymal stem cells, canine stem cells, feline stem cells, bovine stem cells, porcine stem cells, and avian stem cells). In some examples, the cultured cells include mesenchymal stem cells. In some examples, the isolated EVs were released by the cultured cells or tissues.

In some examples, the biological sample is from a plant, such as a plant part, and the methods can be used to isolate plant EVs. In some examples, the biological sample is from plant extract, plant sap, plant juice, plant homogenate, plant lysate, plant filtrate, plant supernatant, plant resin, plant exudate, or plant essential oil. Any suitable plant can be used with the disclosed methods. Exemplary plants include plants belonging to the super family Viridiplantae, such as monocotyledonous and dicotyledonous plants including a fodder or forage legume, ornamental plant, food crop, tree, or shrub, such as Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Doryenium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lo tonus bainesli, Lotus spp., Macro tyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canadensis, Phormium cookianum, Photinia spp., Picea glauca, Pinus spp., Pisum sativum, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, switchgrass, fescue, eucalyptus, a tree, an ornamental plant, a perennial grass and a forage crop. Plant part refers to any part of a plant, including protoplasts, leaves, stems, roots, root tips, anthers, pistils, seeds, embryos, pollens, stamens, ovules, microspores, sporophytes, gametophytes, cotyledons, hypocotyls, flowers, shoots, fruits, tissues, petioles, cells, embryos, callus tissues, and the like.

In some examples, the sample is contacted with the chitin-based material for a period of time. Any suitable period of time can be used, for example, from about 1 hr to about 10 hr (such as from about 1 hr to about 5 hr, from about 2 hr to about 5 hr, or about 2 hr); any period less than about 24 hr, 20 hr, 12 hr, 10 hr, 8, hr, 5 hr, 3 hr, 2 hr, or 1 hr, and/or more than about 10 min, 15 min, 30 min, or 45 min. In some examples, the sample is contacted with the chitin-based material at a suitable temperature, for example, at about 2° C. to about 15° C., such as about 4° C. Following the contacting, the supernatant is removed (e.g., by allowing it to flow through the stationary phase material, or retrieving it from an opening of a container), optionally after applying a magnetic field to collect the chitin-based material if magnetic material is used. Optionally, the stationary phase material may then be washed at least once, with a suitable solution, such as PBS.

In some aspects, the methods further include contacting the chitin-based material contacted with the sample with a thiol-containing reagent that induces self-cleavage of the intein. In some examples, the thiol-containing reagent includes DTT, β-mercaptoethanol, cysteine, or 2-mercaptoethanesulfonate (MESNA). In some examples, the thiol-containing reagent induces cleavage of the intein at its N- or C-terminus, depending on the design of the intein. In some examples, the intein of the fusion protein includes a cysteine or serine at its N-terminus or C-terminus, which can initiate an N—S or N—O acyl shift that converts the amide bond between the intein and the lactC1C2 into a thioester linkage, which can be cleaved by the thiol-containing reagent. In certain examples, the intein is a mini-intein from the Mycobacterium xenopi gyrA gene that has been modified to undergo thiol-induced cleavage at its N-terminus. Additional inteins that can be used in the fusion protein include mxe gyrA intein (Mycobacterium xenopi gyrase A intein), ssp dnaB intein (Synechocystis sp. DnaB helicase intein), ssp dnaX intein (Synechocystis sp. DnaX intein), mth rir1 intein (Methanothermobacter thermautotrophicus ribonucleotide reductase intein), pfu rir1 intein (Pyrococcus furiosus ribonucleotide reductase intein), tli rir1 intein (Thermococcus litoralis ribonucleotide reductase intein), mja rir1 intein (Methanococcus jannaschii ribonucleotide reductase intein), npu dnaB mini-intein (Nostoc punctiforme DnaB mini-intein, engineered form), ssp dnaB mini-intein (engineered variant), ssp dnaB N-terminal cleaving mutant intein, ssp dnaX N-terminal cleaving mutant intein, see vma1 N-terminal cleavage mutant intein, npu dnaB engineered mini-intein (N-cleavage variant), and mth rir1 engineered variant with N-terminal thiol sensitivity.

In some examples, the thiol-containing reagent is contacted with the chitin-based material contacted with the sample for a period of time. Any suitable period of time can be used, for example, from about 5 min to about 5 hr (such as about 10 min, 15 min, 30 min, 45 min, 1 hr, 2 hr, 3 hr, or any range formed therebetween); any period less than about 5 hr, 3 hr, 2 hr, or 1 hr, and/or more than about 5 min, 10 min, 15 min, or 30 min. In some examples, the thiol-containing reagent is contacted with the chitin-based material at a suitable temperature, for example, at room temperature, or at about 2° C. to about 28° C., such as about 10° C. to about 25° C.

In some aspects, the methods further include, after the treatment of the thiol-containing reagent, collecting the supernatant (e.g., by allowing it to flow through the stationary phase material, or retrieving it from an opening of a container), optionally after applying a magnetic field to collect the chitin-based material if magnetic material is used. The supernatant contains the isolated EVs, and may be further processed to remove small molecules and/or enrich the EVs.

In some aspects, the methods further include removing the thiol-containing reagent from the isolated EV composition, by any suitable method known in the art, such as filtration. In some aspects, the methods further include removing lactC1C2 bound to the isolated EVs by any suitable method, such as adding compounds to the isolated EV composition that interrupt the interactions between lactC1C2 and PS. In some examples, the method includes exchanging the buffer of the isolated EV composition with PBS, followed by filtration.

Also provided are compositions and kits, that can be used in the disclosed methods of EV isolation. For example, chitin-based stationary phase materials functionalized with the fusion protein disclosed herein are provided. Kits provided herein may include the fusion protein, and separately a chitin-based stationary phase material. Alternatively, the kits may include a chitin-based stationary phase material functionalized with the fusion protein disclosed herein. In some examples, the kits further include a thiol-containing reagent that induces self-cleavage of the intein.

EVs isolated by the methods disclosed herein can be utilized in a wide range of diagnostic, therapeutic, and biotechnological applications. For example, in some embodiments, EVs isolated from human or animal samples may be used for diagnostic purposes, wherein their molecular cargo (including proteins, lipids, and nucleic acids) reflects the physiological or pathological state of the donor cells, enabling detection and monitoring of diseases such as cancer, neurodegenerative disorders, autoimmune conditions, and cardiovascular diseases. In other embodiments, isolated human or animal EVs may be used as therapeutic or prophylactic agents, or as biocompatible carriers for delivery of therapeutic, prophylactic, or diagnostic agents to a target site within a subject, including across biological barriers such as the blood-brain barrier. In yet other embodiments, EVs obtained through in vitro or ex vivo tissue or cell culturing can be isolated according to the present methods, and the isolated EVs can be used as therapeutic or prophylactic agents, or as biocompatible carriers for delivery of therapeutic, prophylactic, or diagnostic agents to a target site within a subject, including across biological barriers such as the blood-brain barrier. Additionally, EVs isolated from plants using the methods described herein may be used for therapeutic, cosmetic, nutraceutical, or agricultural applications. For example, the isolated plant EVs can serve as natural, biocompatible delivery vehicles for any desired therapeutic, prophylactic, or diagnostic agent.

### EXAMPLES

Novel methods for EV isolation were developed, which utilize the high affinity interaction between lactadherin C1C2 (lactC1C2) and phosphatidylserine (PS) present on EV membranes. A fusion protein including a lactC1C2 domain linked to a chitin-binding domain (CBD) through an intein was constructed, and was used to functionalize chitin-based stationary phase material. The methods yielded EVs with high purity and quality, and can be performed without specialized equipment. Compared to the current gold standard methods, EVs isolated by the present methods are significantly purer and have more well-defined EV morphology.

### Example 1

### Materials and Methods

Construction and Expression of pTXB1-lactC1C2

To amplify the lactC1C2 coding sequence, specific primers were designed based on the lactC1C2 gene sequence retrieved from pJM483 plasmid (Addgene). The NdeI restriction enzyme site sequence was added to the forward primer (5′-ATTAATCATATGTGTTCTACACAGCTGGGCATGGAAGG-3′) (SEQ ID NO: 9) and SapI restriction enzyme site sequence was also added to the reverse primer (5′-ATTAATGCTCTTCCGCAACAGCCCAG CAGCTCCAG-3′) (SEQ ID NO: 10) (Integrated DNA Technologies). The amplification was performed using a thermocycler T-100 (Bio-Rad), with initial denaturation at 98° C. for 1 min, 35 cycles at 94° C. for 60 s, 2 min at 64° C., and 30 s at 72° C., and the final extension was performed at 7° C. for 5 min. The PCR product was analyzed using 1% agarose gel (Thermo Fisher Scientific) electrophoresis. The restriction enzymes NdeI and SpaI (New England Biolabs) were used to cut the insert (lactC1C2), which was then ligated into pTXB1 (New England Biolabs) by a T4 DNA ligase (New England Biolabs) to obtain pTXB1-lactC1C2. Competent E. coli 5a (New England Biolabs) cells were transformed with the final plasmid for amplification. Then, the Plasmid Miniprep kit (QIAGEN) was used for purification.

The pTXB1-lactC1C2 plasmid was transformed into the Lemo21(DE3) strain of E. coli (New England Biolabs) for expression. The transformed bacteria were grown in 1 L Lysogeny broth (LB) (Sigma-Aldrich) containing 100 μg/ml ampicillin (Sigma-Aldrich) with 0.5 mM L-rhamnose (New England Biolabs) at 37° C. in a shaker (250 rpm) until OD₆₀₀ reached 0.5-0.6. Then, the T7 promoter was induced by 0.3 mM isopropyl β-d-1-thiogalactopyranoside (IPTG) (Sigma-Aldrich) at 16° C. for 16 hr. The expression of the lactC1C2-intein-CBD fusion protein was assessed by SDS-PAGE and Western blotting using a lactC1C2 antibody (Thermo Fisher Scientific). The cells were harvested and resuspended in 100 mL of nonionic detergent B-PER (Thermo Fisher Scientific) with EDTA-free protease inhibitor (Thermo Fisher Scientific), and 50 mM Tris-HCl (pH 7.4) (Promega). The crude cell extracts were prepared by sonication for 10 cycles of 30 s (Misonix). The supernatant was separated from the cell debris by centrifugation at 14,000×g for 20 min at 4° C.

Purification and Characterization of lactC1C2 Protein to Test Efficiency of Intein Tag Self-Cleavage

Chitin resin was thoroughly mixed, loaded onto a column, and washed. Crude cell extracts containing the lactC1C2-intein-CBD fusion protein were then loaded onto the resin. The resin was then washed with a column buffer. To separate the lactC1C2 protein from the affinity tag bound to the resin, cleavage was induced by a cleavage buffer containing 50 mM dithiothreitol (DTT) (Sigma-Aldrich) for 16 hr at 4° C., followed by elution with the column buffer. All eluted fractions were collected. OD₂₈₀ was measured for all collected fractions using a microplate reader (SpectraMax M2e; Molecular Devices), and an elution profile was plotted.

The eluted lactC1C2 protein was mixed with an equal volume of 2× Laemmli sample buffer (Bio-Rad) containing 5% 2-mercaptoethanol and heated at 95° C. for 10 min to denature the protein. SDS-PAGE was performed using 12% polyacrylamide gels at 150 V for 60 min, with 1×Tris/glycine/SDS buffer (Bio-Rad). After electrophoresis, the gel was stained using a rapid Coomassie stain (Research Products International) and destained overnight in a buffer containing 40% methanol and 10% acetic acid.

For Western blot analysis, the eluted lactC1C2 protein was transferred onto a 0.45-μm nitrocellulose membrane (Bio-Rad) using the Trans-Blot semi-dry system (Bio-Rad) set at 20 V for 1 hr. The membrane was then blocked in a solution of 5% non-fat dry milk (Santa Cruz Biotechnology) in Tris-buffered saline with 0.1% Tween 20 (TBST) (Thermo Fisher Scientific) with gentle rotation for 1 hr at room temperature, followed by three washes with TBST. The membrane was then incubated with MFG-E8-HRP antibodies (Santa Cruz Biotechnology), specific to lactC1C2, for 1 hr at 4° C. After three washes with TBST, the membrane was imaged using a chemiluminescence imager (Azure 280, Azure Biosystem) with enhanced chemiluminescence (ECL) substrates (Thermo Fisher Scientific) to detect HRP activity.

Functionalization of Chitin Magnetic Beads with lactC1C2-Intein-CBD Fusion Proteins

500 μL-1 mL of crude cell extract containing the lactC1C2-intein-CBD fusion protein was mixed with 200-500 μL of 50-70 μm chitin magnetic beads (New England Biolabs) for 1-2 hr at 4° C., after which the beads were pulled down using a magnet, and the supernatant was collected. The beads were then washed thrice with a washing buffer (NaCl 500 mM, Tris-HCl 20 mM, EDTA 1 mM, pH 8.5) to remove non-specifically bound proteins.

To confirm binding of the fusion protein to the beads and cleavage of the intein tag, the beads were treated with a cleavage buffer containing 50 mM DTT (Sigma-Aldrich) for 16 hr at 4° C., or for 30 min at room temperature. The supernatant and three washes were analyzed by Western blotting or immunoblotting using a lactC1C2-specific antibody.

### Isolation and Characterization of MSC-Derived EVs

Isolation of MSC-derived E1's by chitin beads functionalized with lactC1C2-intein-CBD fusion proteins: Human mesenchymal stem cells (hMSCs) (Lonza) were cultured in alpha minimum essential medium (α-MEM) supplemented with 10% fetal bovine serum (FBS), 1% L-glutamine, and 1% penicillin/streptomycin (Thermo Fisher Scientific) in a T-75 flask in a humidified atmosphere with 5% CO₂ at 37° C. After the cells were grown to approximately 80% confluency, the medium was replaced with serum-free α-MEM medium. The cells were then cultured for 48 hr at 37° C., 5% CO₂. Then, the media were collected and centrifuged at 3000×g for 30 min followed by 10000×g for 30 min to remove cell debris and the apoptotic bodies.

For EV isolation, 100 μL of chitin magnetic beads functionalized with lactC1C2-intein-CBD fusion proteins were mixed with 500 μL of the collected culture media for 2 hr at 4° C. For EV release, intein cleavage was induced using the cleavage buffer containing 50 mM DTT (Sigma-Aldrich) for 16 hr at 4° C. Each fraction (1 mL) containing lactC1C2-EVs was obtained. DTT was removed from the eluted lactC1C2-EVs using a Millipore centrifuge tube (Millipore). Characterization of isolated MSC-derived E1's by Western blotting: EV samples were solubilized at 95° C. for 5-10 minutes and then analyzed by gel electrophoresis. The proteins from the gel were transferred onto a 0.45-μm nitrocellulose membrane (Bio-Rad) using a Trans-Blot semidry system (Bio-Rad). Then, the membrane was blocked with a blocking buffer (5% bovine serum albumin in TBST) with gentle rotation for 1 hr at room temperature. The membrane was then rinsed three times with TBST and then incubated overnight at 4° C. with different primary antibody solutions (1:1000 dilution): CD63, CD81, Alix, and Tsg 101 mAb (Santa Cruz Biotechnology), and lactadherin mAb (Thermo Fisher Scientific). Then, the membrane was washed with TBST to remove any unbound primary antibody and incubated with a secondary antibody, HRP-conjugated mouse IgG secondary antibody (R&D Systems), for half an hour and washed with TBST to remove any unbound secondary antibody. Finally, the membrane was imaged with a chemiluminescence imager (Azure 280) using ECL substrates.

Characterization of isolated MSC-derived E1's by dynamic light scattering (DLS): The size and zeta potential analysis of EVs were conducted to determine the median size and charge of isolated EVs.

### Isolation and Characterization of Plant EVs

Isolation of Arabidopsis thaliana E1's by chitin beads functionalized with lactC1C2-intein-CBD fusion proteins: Arabidopsis thaliana Coumbia-0 (Col-0) seeds were purchased from Arabidopsis Biological Resource Center (Columbus, OH). Plant seeds were sprinkled side-by-side over moist soil and placed at 4° C. for 4 days. After germination, the plants were grown in growth chamber maintained at 22° C. with photoperiod of 16 h light (100 μmol/m²/s) and 8 h of darkness. The plants were further grown for 4 weeks. Set of fifty leaves, freshly weighed at 2.27 g, were cut at petiole and washed with DI water. Once dried, leaves were mixed with 5 mL water, HPLC grade (VWR Chemicals) and disrupted using a high-speed blender (Magic Bullet) for 1 min. The solution was filtered using an 8-μm Whatman filter and centrifuged at 700×g for 30 min at 4° C. (Sorvall ST16R, Thermo Fisher Scientific). The collected fluid was then filtered using a 0.45-μm Nylon filter (Cytvia) and aliquoted and stored at −80° C. for further experiments. Only one freeze-thaw cycle was applied. To isolate EVs, functionalized chitin magnetic beads were resuspended with 500 μL of the fluid collected from Arabidopsis thaliana leaves for 2 hr at 4° C. Then, the beads were pelleted using a magnet and washed thrice with 1×PBS. To release the bound lactC1C2-EVs, the beads were treated with 50 mM DTT for 30 min at room temperature. Then, the beads were pelleted using a magnet, and the supernatant, which contained the released lactC1C2-EVs, was collected. To remove the DTT, the supernatant was passed through 10 kDa Millipore Amicon Ultra Centrifugal Filter (Millipore). The collected supernatants from multiple isolation experiments were resuspended in HPLC grade water (VWR Chemicals) or in cell culture media for further analysis.

Isolation of Arabidopsis thaliana E1's by differential ultracentrifugation: The blended fluid from Arabidopsis thaliana was sequentially subjected to 2000×g and 10,000×g for 30 min at 4° C. (Sorvall ST16R, Thermo Fisher Scientific) in 50 mL tubes. Next, the supernatant was ultracentrifuged at 100,000×g for 1 hr at 4° C. using Optima LE-80K Ultracentrifuge (Beckman Coulter) equipped with Type 70 Ti rotor, and the pellet was washed with HPLC grade water and recentrifuged at 100,000×g for 1 hr at 4° C. Finally, the pellet was resuspended in HPLC grade water for further analysis. For the ultracentrifugation, polycarbonate bottle with cap tubes (Beckman Coulter) were used. Samples were maintained on ice or at 4° C. throughout the isolation process. Following isolation, they were stored frozen at −80° C. To preserve integrity, samples underwent no more than one freeze-thaw cycle.

Characterization of isolated plant EVs by DLS: The size distribution and Zeta potential of the isolated EVs were determined using the NanoBrook 90Plus PALS instrument (Brookhaven Instruments). EVs were resuspended in 1×PBS and filtered using 0.2-μm membrane-filtered HPLC grade water and transferred to a cuvette for measurement.

Characterization of isolated plant E1's by transmission electron microscopy (TEM): 3 μL of an EV sample was added onto a 200-mesh Formvar-carbon coated electron microscopy grid (Ted Pella); after 1 min, the excess liquid was wicked off with a filter paper. The grid was then stained with 2% uranyl acetate (Avantor) for 1 min, after which the excess stain was wicked off. The grid was then washed with HPLC grade water to remove unbound particles, after which the excess water was wicked off. The grid was then kept in a desiccator overnight. Next day, imaging was performed using a transmission electron microscope (Tecnai G² 20 Twin, FEI Company) operated at an appropriate accelerating voltage of 200 kV allowing for high-resolution visualization of vesicle structures. Images were captured with a digital camera system at magnifications ranging from 20,000× to 50,000×.

Characterization of isolated plant EVs by nanoparticle tracking analysis (NTA): NTA was performed using a NanoSight NS300 instrument (Malvern Instruments) to determine the sizes and number of EVs. The instrument was equipped with a 532-nm green laser and capable of detecting particles as small as 20 nm. Samples were diluted to fall within the detection range. For all samples, the camera level was set to 14, frame rate 30 frames per second, detection threshold 5, and syringe pump speed 25.

Characterization of isolated plant EVs by Western blotting: All EV samples were first lysed with RIPA Buffer (Thermo Fisher Scientific) and then diluted with 2× Laemmle sample buffer (Bio-Rad) containing 5% 2-mercaptoethanol BME and heated at 95° C. for 10 min. The samples were then analyzed by SDS-PAGE run at 150 V for 1 hr using 12% polyacrylamide gels and 1× Tris/glycine/SDS buffer (Bio-Rad). The proteins were then transferred to a 0.45-μm nitrocellulose membrane (Bio-Rad) using a semi-dry transfer system (Bio-Rad) at 20 V for 1 hr and 10 min. The membrane was then blocked in 5% non-fat dry milk (Santa Cruz Biotechnology) in TBST with gentle rotation for 1 hr at room temperature. Then the membrane was washed thrice with TBST and incubated with different primary antibodies including CD9, CD63, CD81, TSG101, and negative control calnexin antibodies for 1 hr at 4° C. For analysis using HRP-conjugated primary antibodies, the membrane was then washed thrice with TBST and incubated with ECL substrates for 1 min, and imaged using a chemiluminescence imager (Azure 280). For analysis using non-HRP-conjugated primary antibodies, the membrane was washed thrice with TBST and incubated with an secondary antibody (1:1000 dilution of mouse IgG HRP-conjugated antibody) for 1 hr at room temperature. Then the membrane was washed thrice with TBST and incubated in ECL substrates for 1 min and imaged using a chemiluminescence imager (Azure 280).

### Example 2

Construction of pTXB1-lactC1C2

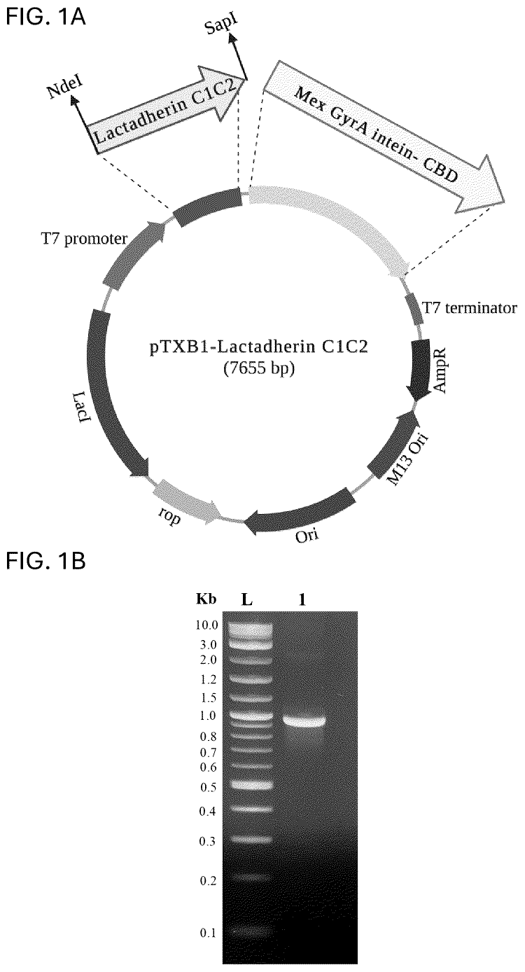

The pTXB1-lactC1C2 plasmid was constructed by inserting the gene encoding a lactC1C2 into apTXB1vector. The gene was cloned from a pJM483 plasmid (Addgene) by polymerase chain reaction (PCR), and the desired PCR product was confirmed by gel electrophoresis (the 0.948 kb band, FIG. 1B). The PCR product and pTXB1 were digested by NdeI and SapI restriction enzymes, followed by ligation of the product into the pTXB1. As shown in FIG. 1A, pTXB1 contains a mini-intein from the Mycobacterium xenopi gyrA gene that has been modified to undergo thiol-induced cleavage at its N-terminus. The lactC1C2 cloned into the NdeI and SapI sites fuses at its C-terminus to the self-cleavable intein tag connected to the chitin-binding domain (CBD). The whole plasmid was also sequenced using Next-Generation Sequencing 3 (Eurofins Genomics). The sequencing results confirmed the correct insertion of the lactC1C2 gene sequence at the intended NdeI and SapI restriction sites within the pTXB1 plasmid. The insert was found to be in the correct reading frame and free of mutations, and with 100% sequence identity to both a reference lactC1C2 sequence from the pJM483 plasmid and CBD sequence, and with 97% sequence identity to a reference intein sequence. Additionally, the plasmid backbone, including the intein and chitin-binding domain (CBD) sequences, was intact and unchanged.

### Example 3

### Expression and Purification of LactC1C2

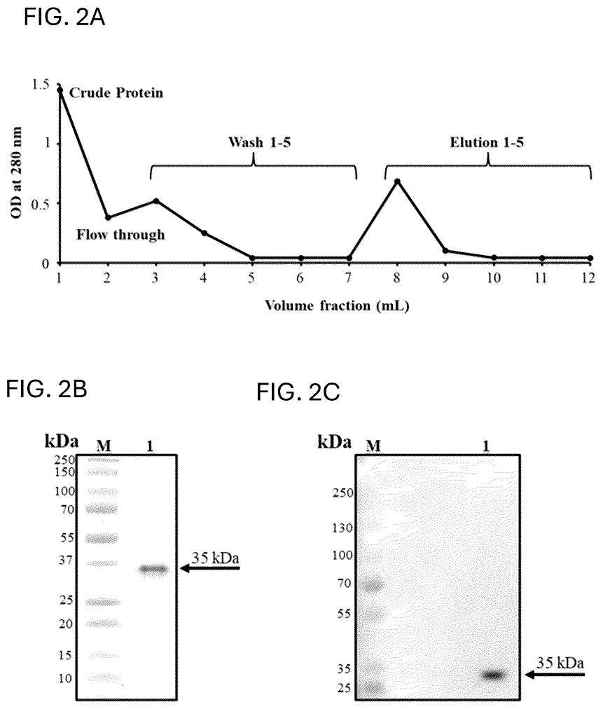

To confirm expression and test the efficiency of intein tag self-cleavage, the fusion protein was expressed from pTXB1-lactC1C2 in transformed E. coli cells, and the crude cell extract was added to chitin resin, followed by DTT treatment to induce intein cleavage. The elution profile (OD₂₈₀) was plotted, which shows the peak at the E1 fraction when most of the proteins were eluted (FIG. 2A). The concentration of the purified lactC1C2 protein was 283±6 μg/mL, as determined by a BCA assay. The gel of SDS-PAGE and the corresponding Western blot membrane incubated with a lactC1C2 antibody both show a single band of ˜35 kDa, corresponding to purified lactC1C2 (FIGS. 2B and 2C).

### Example 4

### Functionalization of Chitin Magnetic Beads

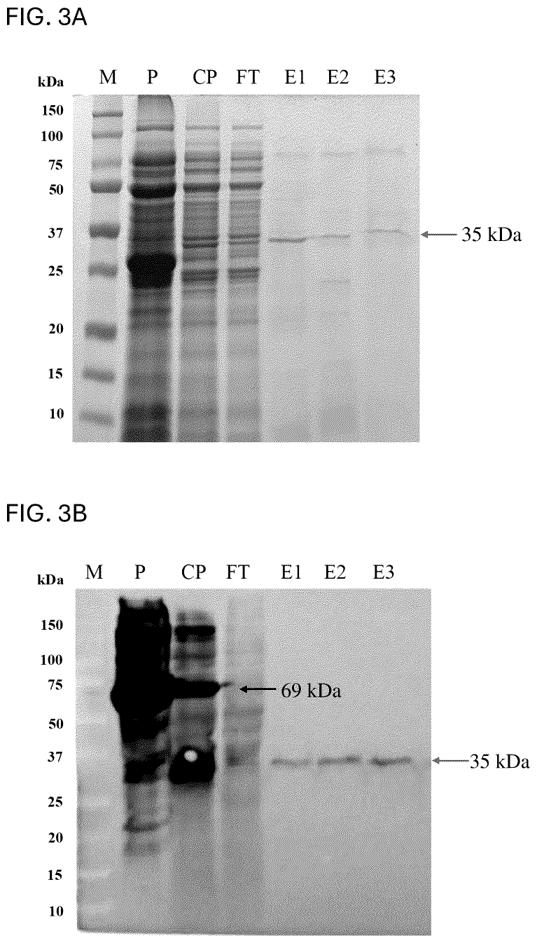

Chitin magnetic beads were contacted with crude cell extracts containing the fusion protein, and were then subjected to DTT treatment. The fusion protein should be about 69 kDa, wherein the intein plus CBD is about 34 kDa, and the lactC1C2 is about 35 kDa. The 69 kDa band confirmed the expression of the fusion protein, and the 35 kDa band confirmed the successful cleavage of the intein tag which released the lactC1C2 (FIGS. 3A and 3B). These results demonstrated that crude lysates can be directly used for functionalization without prior purification of the fusion protein, streamlining the process for generating lactC1C2-functionalized beads.

### Example 5

### Characterization of MSC-Derived EVs

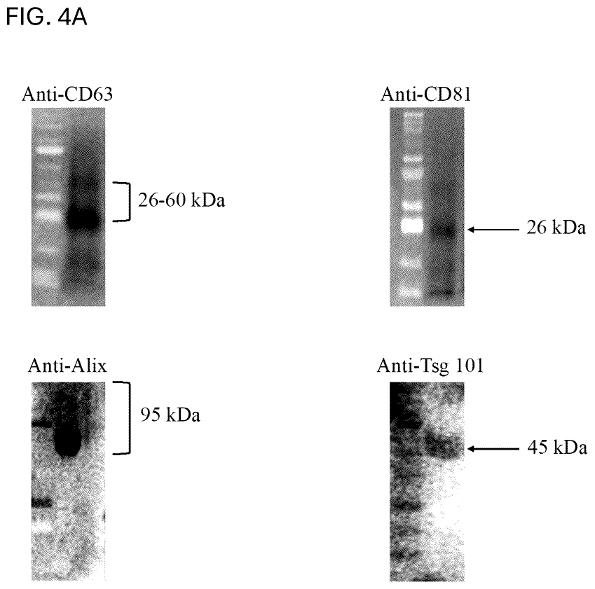

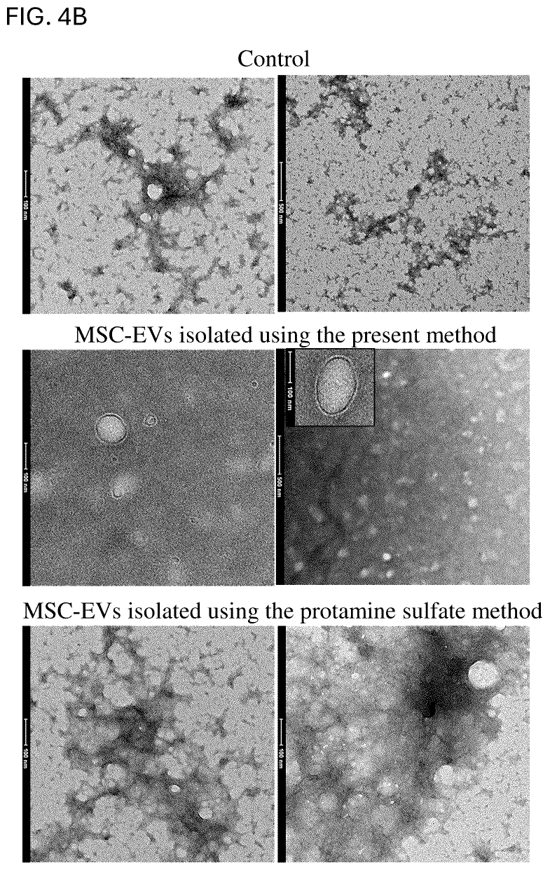

MSC-derived EVs isolated by lactC1C2-functionalized chitin beads were analyzed by Western blotting. As shown in FIG. 4A, the Western blot analysis with EV markers such as CD63, CD81, Alix, and Tsg101 confirmed the enrichment of EV proteins. The concentrations of total proteins in the isolated EV fractions were determined to be 10 μg/mL using a BCA assay. Representative TEM images of the EVs isolated by the present method versus those isolated by the protamine sulfate method are shown in FIG. 4B. These images highlight the markedly cleaner results and more well-defined EV morphology achieved with the present method.

The sizes of the isolated MSC-derived EVs as determined by DLS were 111.21±2 nm, which are within the size ranges of exosomes and microvesicles (K. Kawata et al., “Extracellular vesicles derived from mesenchymal stromal cells mediate endogenous cell growth and migration via the CXCL5 and CXCL6/CXCR2 axes and repair menisci,” Stem Cell Res. Ther., vol. 12, no. 1, pp. 1-13, 2021). The zeta potential of the MSC-derived EVs was measured to be −14.47±2.2 mV.

### Example 6

### Characterization of Plant EVs

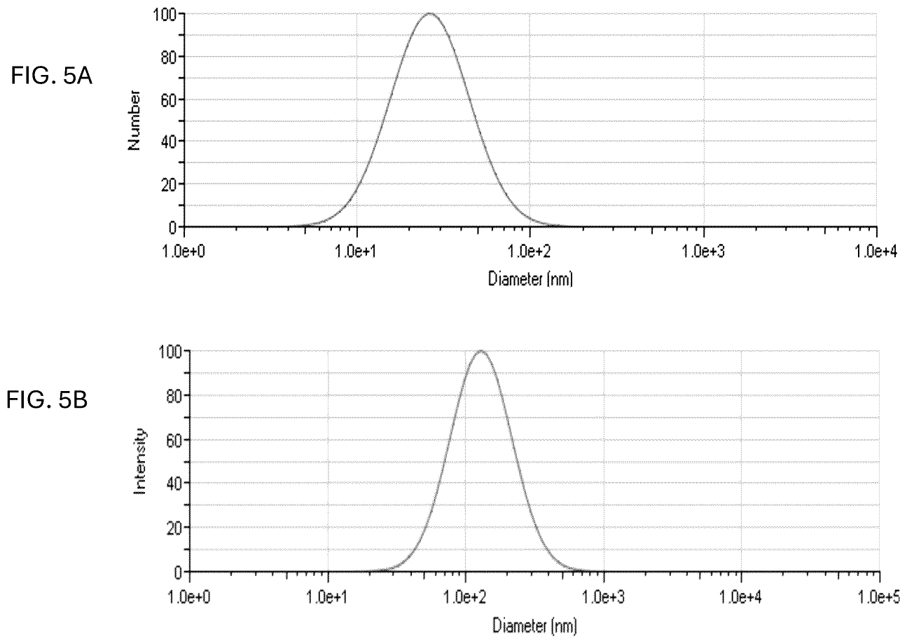

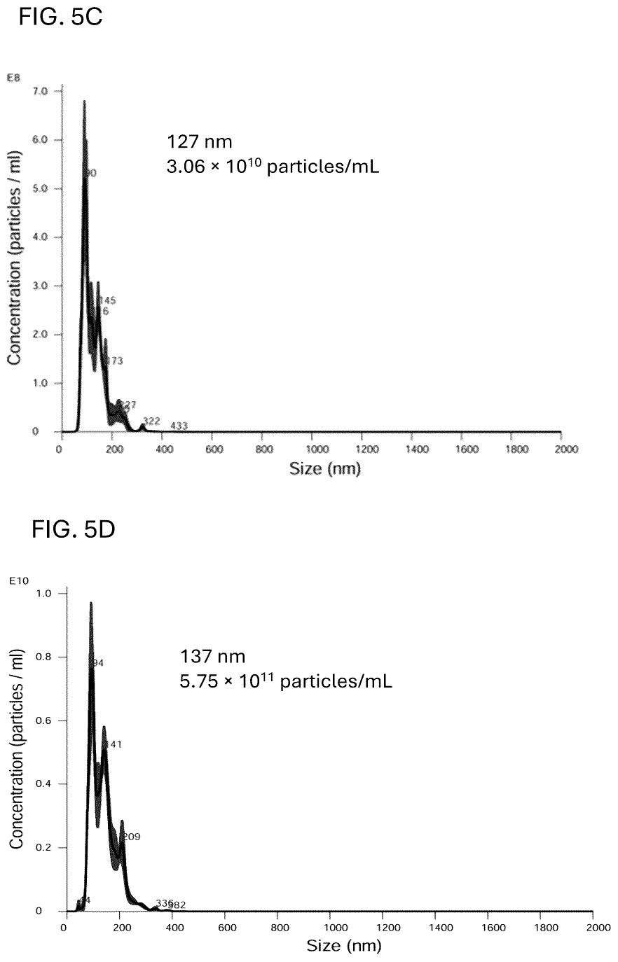

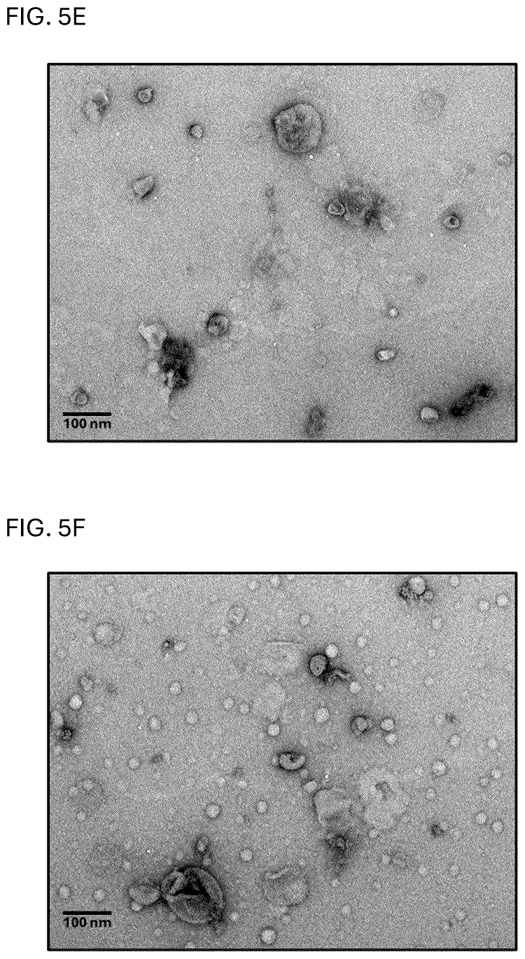

DLS characterization of the isolated EVs revealed a relatively homogeneous size distribution for all EV samples, confirming the successful isolation of EV populations. As shown in FIGS. 5A and 5B, the DLS analysis reported a major intensity-based peak within the EV size range of 50-200 nm, for products isolated by the present method and the differential ultracentrifugation method. The polydispersity index (PDI) was below 0.1, indicating a moderately uniform particle population suitable for downstream applications. In terms of zeta potential of isolated EVs, the charge on all EVs range from −22 to −42 mV. The size distribution of the isolated particles was also evaluated using nanoparticle tracking analysis (NTA), which, unlike DLS that relies on Rayleigh scattering and averages the signal across all particles, tracks individual particles based on Brownian motion and applies the Stokes-Einstein equation for size estimation. As shown in FIGS. 5C and 5D, the NTA reported 127 nm in size and concentration of 3.06×10¹⁰ particles/mL for EVs isolated by the present method. For EVs isolated by the differential ultracentrifugation method, the size reported by NTA was 137 nm and concentration was 5.75×10¹¹ particles/mL.

Transmission electron microscopy (TEM) analysis showed typical cup-shaped morphology of EVs isolated by both methods, after loading the same amount of samples on grids. As shown, EVs isolated by the differential ultracentrifugation method were accompanied by numerous protein aggregates (white dots) (FIG. 5F), whereas EVs isolated by the present method had much higher purity with minimal protein aggregate contaminants (FIG. 5E). This finding is consistent with the NTA results, which show that the particle concentration in purified EVs obtained with the centrifugation method is one order of magnitude higher, than the particle concentration obtained with the present method.

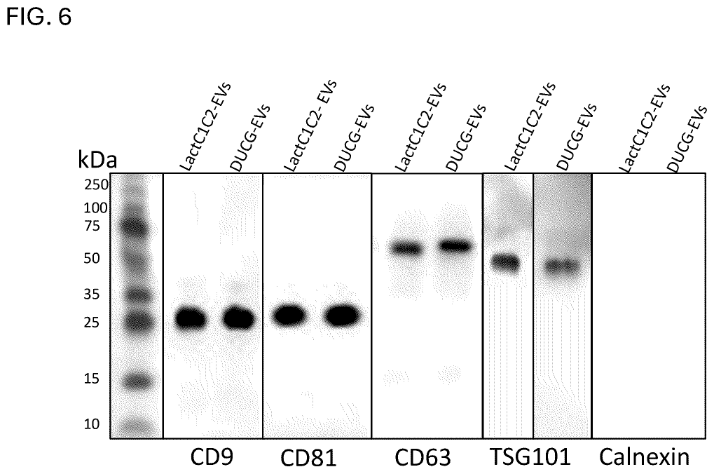

Western blotting was used to verify the presence of EVs by detecting proteins commonly expressed by EVs. As illustrated in FIG. 6, three tetraspanin proteins—CD9, CD63, and CD81—were clearly detected in all EV samples, appearing as bands at approximately 24 kDa, 60 kDa, and 26 kDa, respectively, on the nitrocellulose membrane. The endosomal sorting complex required for transport (ESCRT) associated protein TSG101 was also detected as a distinct band near 50 kDa in all EV samples. Calnexin, an endoplasmic reticulum transmembrane protein used as a negative control, showed no detectable bands in any of the EV samples, confirming the purity of the preparations.

### Example 7

Removing lactC1C2 Bound to EVs

LactC1C2 bound to EVs can be easily removed. One exemplary method involves the use of phosphate-buffered saline (PBS), which will disrupt the interaction between lactC1C2 and PS. Thus, PBS may be used to remove lactC1C2 bound to EVs after the lactC1C2-bound EVs are released from the intein-CBD tag.

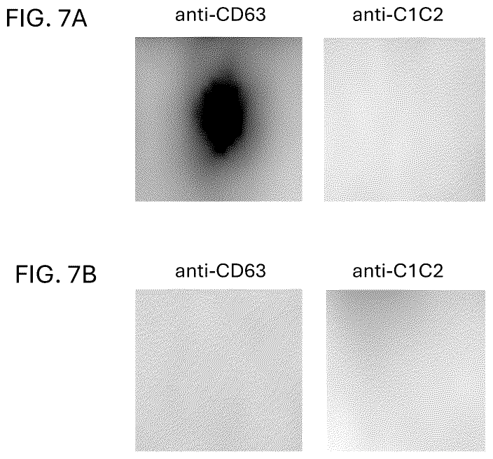

Chitin magnetic beads functionalized with the fusion protein were used to capture EVs from conditioned medium of MSC culture or extract of plant leaves, then washed with 1×PBS for 30 min at room temperature. The beads were collected by a magnet and the supernatant was collected and immunoblotted with an anti-CD63 mAb and anti-lactC1C2 mAb. As shown in FIG. 7A, there is a strong signal from the blotting with the anti-CD63 mAb indicating the presence of EVs in the fluid, while there is no signal from the blotting with the anti-lactC1C2 mAb, confirming that the EVs were not associated with lactC1C2. The beads were further washed with PBS for 30 min at room temperature, aggregated by a magnet, and the supernatant collected and immunoblotted. As shown in FIG. 7B, no signal was detected in the anti-CD63 or anti-lactC1C2 blots.

We claim:

1. A method of isolating extracellular vesicles (EVs) from a sample, comprising contacting the sample with a chitin-based stationary phase material functionalized with a fusion protein, wherein the fusion protein comprises i) a lactadherin C1C2 domain, ii) a chitin-binding domain (CBD), and iii) an intein positioned between the lactadherin C1C2 domain and the CBD.

2. The method of claim 1, further comprising contacting the chitin-based stationary phase material contacted with the sample with a thiol-containing reagent that induces self-cleavage of the intein.

3. The method of claim 1, wherein the chitin-based stationary phase material comprises chitin beads.

4. The method of claim 3, wherein the chitin beads are magnetic.

5. The method of claim 1, wherein the lactadherin C1C2 domain comprises an amino acid sequence that is at least 80% identical to SEQ ID NO: 1, or is encoded by a nucleic acid comprising a nucleic acid sequence that is at least 80% identical to SEQ ID NO: 5.

6. The method of claim 1, wherein the CBD comprises an amino acid sequence that is at least 80% identical to SEQ ID NO: 2, or is encoded by a nucleic acid comprising a nucleic acid sequence that is at least 80% identical to SEQ ID NO: 6.

7. The method of claim 1, wherein the intein comprises an amino acid sequence that is at least 80% identical to SEQ ID NO: 3, or is encoded by a nucleic acid comprising a nucleic acid sequence that is at least 80% identical to SEQ ID NO: 7.

8. The method of claim 1, comprising, from N-terminal to C-terminal, the lactadherin C1C2 domain, the intein, and the CBD domain.

9. The method of claim 1, wherein the fusion protein comprises an amino acid sequence that is at least 80% identical to SEQ ID NO: 4, or is encoded by a nucleic acid comprising a nucleic acid sequence that is at least 80% identical to SEQ ID NO: 8.

10. The method of claim 1, wherein the sample is a biological sample.

11. The method of claim 10, wherein the biological sample is from a human, an animal, a plant, or a microorganism.

12. The method of claim 10, wherein the biological sample comprises a bodily fluid.

13. A fusion protein comprising i) a lactadherin C1C2 domain, ii) a chitin-binding domain (CBD), and iii) an intein positioned between the lactadherin C1C2 domain and the CBD.

14. The fusion protein of claim 13, comprising, from N-terminal to C-terminal, the lactadherin C1C2 domain, the intein, and the CBD domain.

15. The fusion protein of claim 13, comprising the amino acid sequence of SEQ ID NO: 4.

16. A nucleic acid encoding the fusion protein of claim 15.

17. The nucleic acid of claim 16, comprising the nucleic acid sequence of SEQ ID NO: 8.

18. A vector comprising the nucleic acid of claim 16.

19. A chitin-based stationary phase material functionalized with the fusion protein of claim 13.

20. A kit comprising:
the fusion protein of claim 13, and a chitin-based stationary phase material; or
a chitin-based stationary phase material functionalized with the fusion protein of claim 13.

21. The kit of claim 20, further comprising a thiol-containing reagent that induces self-cleavage of intein.
